(19) United States
(12) Patent Application Publication
(10) Pub. No.: US 2019/0111024 A1
(43) Pub. Date: Apr. 18, 2019

(54) Method of Treatment of Post-Traumatic Stress Disorder

(71) Applicant: Juan Tejada-Duran, South Lawrence, MA (US)

(72) Inventor: Juan Tejada-Duran, South Lawrence, MA (US)

(21) Appl. No.: 15/730,935

(22) Filed: Oct. 12, 2017

(51) Int. Cl. A61K 31/4045; A61K 31/195; A61K 9/00; A61K 31/07; A61K 31/4415; A61K 31/51; A61K 31/525; A61K 31/592; A61K 31/355; A61K 31/375; A61K 31/714; A61K 31/4188; A61K 31/28

(52) U.S. Cl. CPC A61K 31/4045; A61K 31/195; A61K 9/0053; A61K 31/07; A61K 31/4415; A61K 31/51; G01N 33/48; A61K 31/592; A61K 31/355; A61K 31/375; A61K 31/714; A61K 31/4188; A61K 31/28; A61K 31/525

(57) ABSTRACT

A method of treating post-traumatic stress disorder is provided. The method includes administering a composition of amino acids, melatonin, and vitamins, and then tracking, through testing and the like, a progression of the treatment. Typically, but not necessarily, the composition is administered orally in the form of pills, powder, pre-mixed food items, and the like.

### BACKGROUND OF THE INVENTION

### Field of the Invention

The present invention relates generally to a medicinal treatment of a stress disorder. More particularly the present invention relates to method of administering the medicinal treatment in the form of a composition, and tracking progress in a patient suffering from post-traumatic stress disorder.

Almost one hundred years ago Dr Asbjorn Folling discovered that the absence of the enzyme phenylalanine hydroxylase resulted in a condition named phenylketonuria due to a buildup and increase of the levels of phenylalanine. I have come to the conclusion that it is not the increased amounts of phenylalanine that effects the CNS in the body; it more the lack of the metabolic pathway in breaking phenylalanine into tyrosine, dopamine, norepinephrine and epinephrine and other essential chemicals that cause mentally ill diseases. The codon Uracil-Uracil-Uracil also known as the phenylalanine amino acid; that is the first amino acid in the genetic code discovered by Mr Marshall Nuremberg; is the pivotal source of CNS energy. Phenylalanine sustains our psychological and physical wellbeing. Phenylalanine I have found is effective in treating not only Post Traumatic Stress Disorder (PTSD), but also very effective in the treatment of severe psychotic episodes in Bipolar Disorder, various types of Depression, Schizophrenia, Parkinson's and Alzheimer's Disease.

The following is this exact pathway that phenylalanine and each resulting chemical breaks down into the final product, which is epinephrine. This breakdown pathway further demonstrates that when the breakdown pathway is not in order, and the essential chemicals are lacking or not formed. The pathway is not functioning correctly or exhausted therefore diseases such as PTSD and others occur.

Phenylalanine->Tyrosine->DOPA->Dopamine->Norepinephrine->Epinephrine.

### Description of Related Art

Individuals responding to stressful events can experience one, two, or three of the following stages: (1) stage one, mobilization of energy; (2) stage two, exhaustion or consuming energy; and (3) stage three, draining energy stores.

In stage one, the mobilization of energy, the body responds to stress through a release of Adrenalin and a fight or flight response. Symptoms of this stage include increased heart rate and blood pressure, rapid breathing, sweating, decreased digestion rate and indigestion.

If there is no relief from stage one, the individual enters phase two, exhaustion or consuming energy. Here, the body will begin to release stored sugars and use up fats. Symptoms of this stage include feeling driven, feeling pressured, tiredness and fatigue, increased smoking, coffee drinking and/or alcohol consumption, anxiety, memory loss, and acute illnesses such as colds and flu.

If either the stressful situation is not resolved, or the individual's reaction to the situation is not changed, the individual enters phase three, draining energy sources. Here, the individual becomes chronically stressed, and the body's need for energy resources outpaces its ability to produce them. Symptoms of this stage include serious illnesses such as heart disease, ulcers, and mental illness, as well as insomnia, errors in judgment, and personality changes.

At present, patients suffering from stress related disorders are treated for the symptoms of stress by the use of pharmaceutical compositions containing drugs such as anxiolytics or sometimes with beta-blockers. Anxiolytics frequently used include drugs such as benzodiazepines, diazepam being a specific example. The beta-blocking drugs used for treatment of such patients include propranolol, and the like. Other treatment methods include the use of an inhibitor of norepinephrine, or a combination serotonin and norepinephrine inhibitor. Examples include Venlafaxine (Effexor®), a norepinephrine inhibitor or Duloxetine (Cymbalta®) which inhibits serotonin and norepinephrine.

Veterans in particular are quite susceptible to stress disorders including post-traumatic stress disorder (hereinafter “PTSD”). PTSD has lead to alarming numbers of suicides and destructive behavior by veterans, and this problem continues unabated. While certain medications as noted above may be helpful, medical nutrient supplementation is a more naturally and gentle form of treatment with fewer side-effects, and may be more desirable in many situations.

Therefore, what is needed is a treatment method for treating and monitoring PTSD which can help alleviate and cure symptoms of the disease.

### SUMMARY OF THE INVENTION

The subject matter of this application may involve, in some cases, interrelated products, alternative solutions to a particular problem, and/or a plurality of different uses of a single system or article.

In one aspect, a method of treating post-traumatic stress disorder is provided. The method involves administering a medicinal composition to a patient suffering from post-traumatic stress disorder. The composition comprises a quantity of all twenty essential amino acids, melatonin, and a complex comprising multiple vitamins and minerals. This administration of the composition is repeated daily (every 24 hours) and may be spread in doses throughout the day and/or night. The method further involves testing the patient. The testing may comprise taking a biological sample from the patient, analyzing the sample for a biological marker of post-traumatic stress disorder, and comparing the analyzed sample to a previously analyzed sample. By taking and repeating the testing, a progress, or lack thereof, of the patient may be tracked to observe how effective the treatment method is. For example, the testing may involve identifying an increase or decrease in at least one of cortisol, dopamine, and norepinephrine compared to a previously analyzed sample.

In another aspect, a method of treating post-traumatic stress disorder is provided. The method involves administering a composition to a patient suffering from post-traumatic stress disorder. The composition consists of a quantity of all twenty essential amino acids, including 500 mg of D-L-phenylalanine and 500 mg of L-tyrosine, as well as 5 mg of melatonin, and a vitamin and mineral complex consisting of vitamins C, B1, B2, B3, B5, B6, B9, B12, biotin, A, E, D2 (or D3), K, potassium, iodine, selenium, borate, zinc, calcium, magnesium, manganese, molybdenum, beta carotene, and iron. This administration of the composition is repeated daily (every 24 hours) and may be spread throughout the day and/or night. The method further involves testing the patient. The testing may comprise taking a biological sample from the patient, analyzing the sample for a biological marker of post-traumatic stress disorder, and comparing the analyzed sample to a previously analyzed sample. By taking and repeating the testing, a progress, or lack thereof, of the patient may be tracked to observe how effective the treatment method is.

### BRIEF DESCRIPTION OF THE DRAWINGS

FIG. 1 provides a flow chart of an embodiment of use of the present invention.

### DETAILED DESCRIPTION

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments.

Generally, the present invention concerns a method of treatment of post-traumatic stress syndrome (“PTSD”) which includes administration of a composition, and subsequent testing of progress. The administration of the composition is repeated, typically indefinitely, though may stop eventually in some cases. Typically, administration of the composition is continued daily, and at least until tested treatment parameters have reached a desired level. The testing may be any testing capable of determining factors indicating PTSD, which may include biological factors, or testing relating to a patient's mental state. This testing may also be used to increase or decrease the quantity of the composition or the frequency of its administration depending on results. For example, if the results are not indicative of a desired progress, an amount of the components may be increased, and if the results are indicative of the desired progress, the amount may be decreased.

In one embodiment, the present composition comprises a quantity of all twenty essential amino acids. In a particular embodiment, the composition may have a quantity of D-L-phenylalanine of 500 mg. In another particular embodiment, the composition may have a quantity of L-tyrosine of 500 mg. In still another embodiment, the composition may have both 500 mg of D-L-phenylalanine and 500 mg of L-tyrosine. In one embodiment, the composition may have an equal mass of each of the 20 amino acids. In another embodiment, the composition may have varied amounts of the different amino acids. The composition may further comprise a quantity of melatonin, such as 3 mg or 5 mg. Finally, the composition provides a supplement of multiple vitamins and minerals, such as those commonly found in a multivitamin complex. Examples of the vitamin and mineral composition include, but are not limited to vitamins C, B1, B2, B3, B5, B6, B9, B12, biotin, A, E, D2 (or D3), K, potassium, iodine, selenium, borate, zinc, calcium, magnesium, manganese, molybdenum, beta carotene, and/or iron. In a particular embodiment, a large dosage of vitamin B12 may be provided which may aid in uptake of the amino acids and accommodate increased B12 needs of the PTSD-suffering patient.

In a further embodiment, the composition may be administered in combination with additional medication such as anxiolytics and/or beta-blockers. In a particular embodiment, the additional medication may be integrated into the composition such that the additional medication is taken at the same time as the other components of the composition. In another embodiment the additional medication may be separated from the composition administration, such as, for example a separate pill.

The composition may be administered once daily, or many times daily, depending on embodiment. In one embodiment, a patient may take the composition orally at night time. In another embodiment, the patient may take the composition upon waking or after breakfast. In still another embodiment, the patient may take dosages of the composition three times daily, five times daily, six times daily, and the like. In embodiments of taking multiple dosages during a day, the composition may be uniform, or may vary throughout the day. For example, the composition may be amino-acid heavy for a morning dosage, and melatonin heavy for an evening dosage to aid in sleep. This may also be reversed and adjusted. In another embodiment, the composition taken multiple times in the day may be the same composition. Administration of the composition is generally by oral administration, but in other embodiments may be injected subcutaneously, intravenously, and the like.

Compositions of the present invention used for oral administration may be in any number of different forms. In one embodiment, the composition may be in the form of a pill or a plurality of pills. In another embodiment, the composition may be powdered to be mixed into a fluid or a food. In yet another embodiment, the composition may be sold pre-mixed into a food or drink item.

Examples of different types of testing may include biological marker testing, and/or psychological testing. In many cases, those suffering from PTSD have identifiable biological markers that can be observed through testing of blood, saliva, urine, and the like. For example, in many cases, PTSD patients may have abnormal (elevated, decreased, or volatile and inconsistent) levels of cortisol, dopamine, norepinephrine, neuropeptide S, and the like. Also, PTSD patients may demonstrate increased insulin resistance. Many other markers known or discovered may also be observed to track the presence of PTSD. The testing may compare levels of a biological marker to an expected value or range from a healthy individual, and/or the levels, over time, can be tracked to track overall PTSD treatment efficacy and progression.

In many cases, psychological testing may be used instead of or in addition to the biological marker testing. Psychological testing may include answering a question or series of questions designed to indicate certain symptoms of PTSD. Further, psychological testing may involve interviews with trained staff, activities, and the like. The testing may be used to detect a presence of PTSD or, in many instances, may be used over time to track the progress of a patient suffering from PTSD. Psychological testing may be performed on a computer, and/or with supervision from a trained staff member.

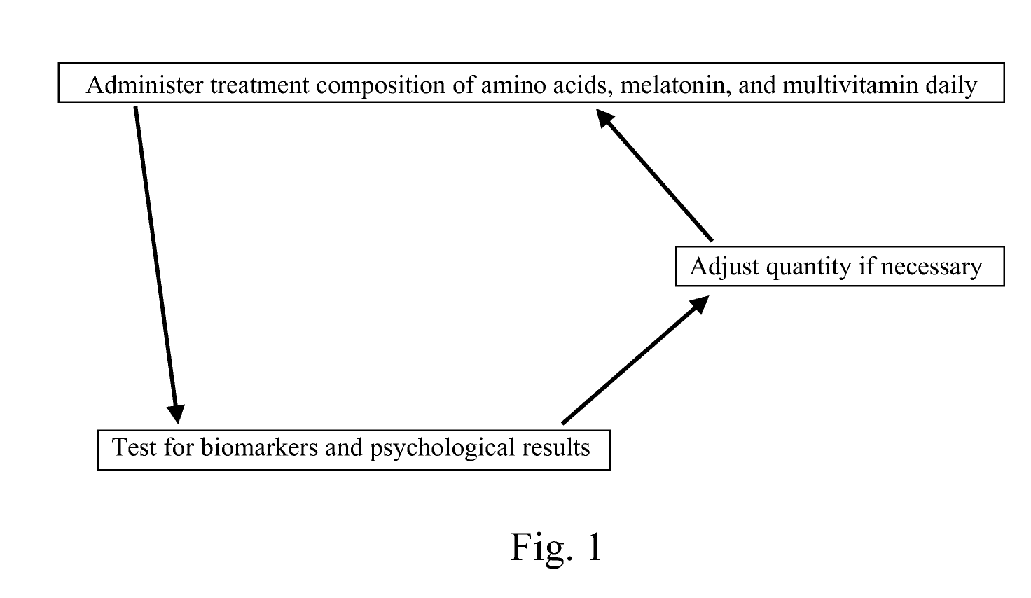

Turning now to FIG. 1, an embodiment of treatment of PTSD of the present invention is provided. The process begins with identification of a patient with PTSD, and administering a composition comprising amino acids, melatonin, and multivitamins daily. Initially, a base line test may be taken to evaluate the biomarkers and/or psychological status of the patient. Over a period of time of the administration, one or more tests is performed to track the biomarkers and/or psychological status. Depending on the progress and changes noted during testing, the composition dosage may be increased, decreased, or kept the same. In many cases, the testing results will show positive progress in the PTSD and the composition administration frequency and amount can be maintained as a preventative measure, or decreased as the patient is better able to manage the PTSD.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

What is claimed is:

1. A method of treating post-traumatic stress disorder comprising the steps of:
administering a composition to a patient suffering from post-traumatic stress disorder, the composition comprising:
a quantity of all twenty essential amino acids;
a quantity of melatonin; and
a complex comprising multiple vitamins and minerals;
wherein the step of administering is repeated every 24 hours; and
testing the patient, the step of testing comprising taking a biological sample from the patient, analyzing the sample for a biological marker of post-traumatic stress disorder, and comparing the analyzed sample to a previously analyzed sample.

2. The method of claim 1 wherein the quantity of amino acids comprises 500 mg of D-L-phenylalanine.

3. The method of claim 2 wherein the quantity of amino acids comprises 500 mg of L-tyrosine.

4. The method of claim 3 wherein the composition comprises 5 mg of melatonin.

5. The method of claim 4 wherein the complex of vitamins and minerals comprises vitamins C, B1, B2, B3, B5, B6, B9, B12, biotin, A, E, D2, K, potassium, iodine, selenium, borate, zinc, calcium, magnesium, manganese, molybdenum, beta carotene, and iron.

6. The method of claim 5 wherein the step of administering comprises separating the composition into three equal quantities, and administering a first quantity in the morning, a second quantity at mid-day, and a third quantity at night.

7. The method of claim 5 wherein the step of administering comprises separating the composition into three unequal quantities, and administering a first quantity in the morning, a second quantity at mid-day, and a third quantity at night.

8. The method of claim 5 wherein the step of administering comprises separating the composition into six quantities, and administering one of the six quantities six times per day.

9. The method of claim 5 wherein the testing step comprises testing a level of each of cortisol, dopamine, and norepinephrine.

10. The method of claim 9 wherein the testing step comprises identifying an increase or decrease in at least one of cortisol, dopamine, and norepinephrine compared to the previously analyzed sample.

11. The method of claim 5 wherein the testing step comprises measuring an insulin resistance of the patient.

12. The method of claim 11 wherein the testing step comprises identifying a decrease in measured insulin resistance compared to the previously analyzed sample.

13. The method of claim 5 wherein the testing step comprises comparing the biomarker to an expected level of a subject not suffering from post-traumatic stress disorder.

14. The method of claim 5 wherein the quantity of amino acids comprises 500 mg of each of the twenty amino acids.

15. A method of treating post-traumatic stress disorder comprising the steps of:
administering a composition to a patient suffering from post-traumatic stress disorder, the composition consisting of:
a quantity of all twenty essential amino acids, including 500 mg of D-L-phenylalanine and 500 mg of L-tyrosine;
5 mg of melatonin; and
a vitamin and mineral complex consisting of vitamins C, B1, B2, B3, B5, B6, B9, B12, biotin, A, E, D2, K, potassium, iodine, selenium, borate, zinc, calcium, magnesium, manganese, molybdenum, beta carotene, and iron;
wherein the step of administering is repeated every 24 hours; and
testing the patient, the step of testing comprising taking a biological sample from the patient, analyzing the sample for a biological marker of post-traumatic stress disorder, and comparing the analyzed sample to a previously analyzed sample.

16. The method of claim 15 wherein the testing step comprises testing a level of each of cortisol, dopamine, and norepinephrine.

17. The method of claim 16 wherein the testing step comprises identifying an increase or decrease in at least one of cortisol, dopamine, and norepinephrine compared to the previously analyzed sample.

18. The method of claim 15 wherein the testing step comprises measuring an insulin resistance of the patient.

19. The method of claim 18 wherein the testing step comprises identifying a decrease in measured insulin resistance compared to the previously analyzed sample.

20. The method of claim 5 wherein the step of administering comprises separating the composition into multiple equal quantities, and administering each of the equal quantities throughout the day.
